(19) United States
(12) Patent Application Publication
(10) Pub. No.: US 2009/0268870 A1
(43) Pub. Date: Oct. 29, 2009

(54) Mobile radiation treatment vehicle and method

(76) Inventor: Mark E. Pomper, Miami Beach, FL (US)

Correspondence Address: MALLOY & MALLOY, P.A., 2800 S. W. Third Avenue, Historic Coral Way, Miami, FL, 33129, US

(21) Appl. No.: 12/454,469

(22) Filed: May 18, 2009

Related U.S. Application Data
(63) Continuation of application No. 10/588,739, filed on Nov. 20, 2006.
(60) Provisional application No. 60/449,676, filed on Feb. 24, 2003.

(51) Int. Cl. A61N 5/10; G21F 3/00; A61G 99/00

(52) U.S. Cl. 378/65; 250/515.1; 296/24.38

(57) ABSTRACT

A mobile radiation treatment vehicle is disclosed including, a patient treatment compartment having at least one radiation shield member, the at least one radiation shield member positioned to prevent at least a portion of radiation emitted from a treatment device from passing through an interior of the patient treatment compartment to an outside area, the treatment device capable of emitting radiation used in connection with radiation therapy and positioned in the patient treatment compartment, and a shielded partition member positioned in the patient treatment compartment and proximate to the treatment device, the shielded partition member positioned to reduce or prevent exposure to a user from radiation emitted from the treatment device during patient treatment.

### CLAIM OF PRIORITY

The present application is a continuation application of previously filed, now pending application having Ser. No. 10/588,739, filed on Aug. 4, 2006, which claims priority to PCT application having Serial No. PCTUS04005354 which was filed on Feb. 24, 2004, which claims priority to U.S. Provisional Application Ser. No. 60/449,676, filed on Feb. 24, 2003, the contents of all of which are incorporated herein by reference.

### FIELD OF THE INVENTION

The present invention relates generally to radiation therapy. More specifically, the present invention relates to a mobile radiation treatment vehicle and method for providing radiation therapy to a patient.

### BACKGROUND OF THE INVENTION

Radiation therapy has been in use for the treatment of cancer and other diseases for approximately 100 years. As early as 1897, it was concluded that x-rays could be used for therapeutic as well as diagnostic purposes, and in 1912, Marie Curie published the “Theory of Radioactivity.” The investigation of x-ray radiation for patient therapy moved into the clinical routine in the early 1920s.

Since the first uses of radiation to treat cancer, important changes have been made in the field and numerous developments have been accomplished, including: the generation of higher energy radiation beams for more effective cancer treatment; the development of versatile linear accelerator and patient table designs to enable radiation to be delivered to the cancer from a variety of angles and directions; the implementation of “multi-leaf” collimators (lead shutters) and other beam shaping devices for precision control and shaping of the radiation beam; the use of CT, PET, MR and other image data sets to create three-dimensional planning models to accurately guide treatment; and the implementation of networked computers to track radiation treatment sessions and patient dose calculations (both planned and accumulated).

The ultimate goal of all of the above changes, developments and improvements is the effective destruction of cancer tissue while delivering a minimal dose of radiation to adjacent healthy tissues. Another goal is to make the treatment easier and shorter for the patient to sustain and the physicians and other healthcare professionals to perform.

However, with all the advances in radiation treatment, there are urgent needs that have not been met. There are many patients in need of radiation therapy that are unable to travel to treatment centers. Mobile X-ray units have been in use for years and provided physicians an important diagnostic tool for evaluating patients unable to travel to receive an x-ray. Radiation therapy is much more prohibitive due to the prolonged periods in which the radiation source is active. The need for a safe, effective mobile radiation therapy system has been needed and not met. The present invention fulfills the need for mobile radiation therapy.

### SUMMARY OF THE INVENTION

The present invention eliminates the above-mentioned needs by providing a mobile radiation treatment vehicle and a method for providing radiation treatment.

In accordance with the present invention, there is provided a mobile radiation treatment vehicle including, a patient treatment compartment, the patient treatment compartment for housing a treatment device capable of emitting radiation used in connection with radiation therapy, and a shielded partition member positioned in the patient treatment compartment and proximate to the treatment device, the shielded partition member positioned to reduce or prevent exposure to a user from radiation emitted from said treatment device during patient treatment.

The present invention is further directed to a method for providing radiation therapy including, preparing a mobile radiation treatment vehicle having: a patient treatment compartment having at least one radiation shield member, at least one radiation shield member positioned to prevent at least a portion of radiation emitted from a treatment device from passing through an interior of the patient treatment compartment to an outside area; the treatment device capable of emitting radiation used in connection with radiation therapy and positioned in the patient treatment compartment; and a shielded partition member positioned in the patient treatment compartment and proximate to the treatment device, the shielded partition member positioned to reduce or prevent exposure to a user from radiation emitted from the treatment device during patient treatment, providing access to an interior area of the patient treatment compartment to a patient, securing the treatment device in a position relative to the patient, providing radiation therapy to the patient, and shielding the user from at least a portion of the radiation emitted from the treatment device.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is an isometric view of the preferred embodiment of the present invention.

FIG. 2 is a top view of the interior of the patient treatment compartment of the present invention shown in FIG. 1.

FIG. 3 is a front perspective view of components illustrated in FIG. 2.

FIG. 4 is a flow-chart diagram of the system of the present invention.

Like reference numerals refer to like parts throughout the several views of the drawings.

### DETAILED DESCRIPTION OF THE PREFERRED EMBODIMENT

The present invention relates to a vehicle and method for providing mobile radiation therapy. Radiation therapy is typically used to treat cancer. The term, “cancer” is meant to describe any physiological condition in which cells are not being regulated by normal mechanisms. Additional conditions for therapy may include, but would not be limited to: sarcomas, lymphomas, keloids, benign recalcitrant dermitidies, warts, calluses, arthritis, ulcers, pterygium, psoriasis and the like. The present invention is intended and suitable for treatment of any medical condition in which radiation therapy would be medically prescribed and beneficial. The units conventionally utilized in radiation therapy are typically housed in hospitals or radiation therapy centers. The present invention provides for a radiation therapy unit that has been adapted for transport and treatment in a mobile therapy unit.

The source of the radiation therapy unit may be any unit such that a therapeutic amount of radiation is delivered to a patient in a safe and medically acceptable manner. Those skilled in the art are well versed in the time parameters for therapy as well as the conventionally utilized dosages of radiation. Radiation therapy units may be used to deliver up to 35 mV of radiation. For superficial treatments, levels up to 250 kV may be used. The present invention is envisioned to provide a system for providing treatment in any therapeutic range of radiation therapy.

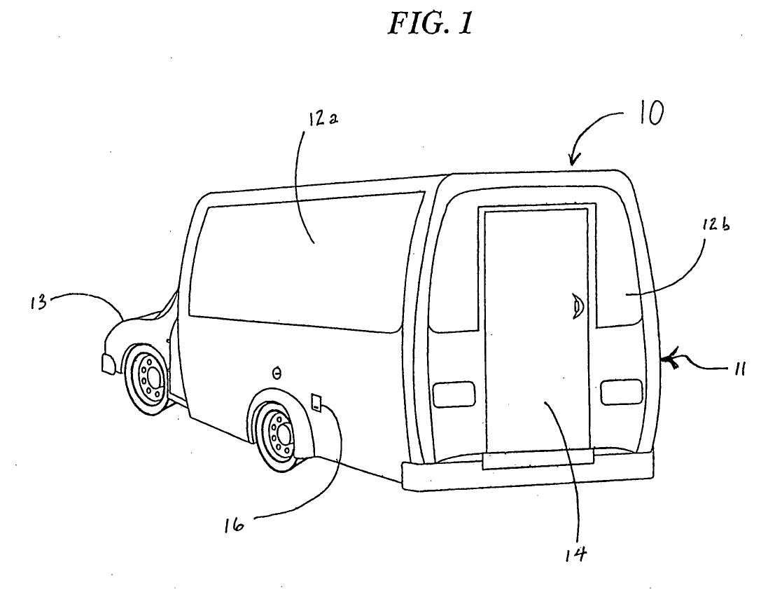

In one embodiment, a vehicle (described in detail below with reference to FIG. 1) is lined with lead sufficient to prevent exposure to radiation outside the treatment area. The lead may be 1/32 to 12 inches in thickness. In a preferred embodiment, the lead shielding is 1/16 inch thick. The lining is installed within the mobile transport unit in a manner as is conventionally known in order to prevent any radiation from escaping the treatment area within the vehicle. The interior of the vehicle is also suitably equipped with a lead lined area for the operator of the therapy machine to safely administer the radiation therapy, as discussed below. The operation area is further constructed with a conventionally used radiation-inhibiting window for the operator to observe the patient during therapy.

Referring now to FIG. 1, the preferred embodiment of the present invention is illustrated as mobile radiation treatment vehicle 10. Mobile radiation treatment vehicle 10 includes a patient treatment compartment 11, which is adjacent to vehicle cabin 13. Vehicle cabin 13 houses the driving controls for mobile radiation treatment vehicle 10.

Patient treatment compartment 11 has an exterior that preferably incorporates at least one radiation shield member 12a, and more preferably additional radiation shield members such as radiation shield member 12b. Radiation shield members 12a and 12b can be formed from any one of a number of materials known in the art that at least partially prevent the passage of radiation, including but not limited to lead, aluminum, alloys of lead, polymers (such as acrylic), concrete, and fiberglass. The thickness of radiation shield members 12a and 12b is dependent upon the material used to form radiation shield members 12a and 12b. Alternatively, in another embodiment of the present invention, radiation shield members 12a and 12b are not required if precautions are undertaken to prevent the public from approaching too close to mobile radiation treatment.

Patient treatment compartment 11 further includes a power adapter 16. Power adapter 16 can be engaged to provide direct line electrical power to the system of the present invention without the use of the resources of mobile radiation treatment vehicle 10. Patient treatment compartment 11 additionally incorporates an access door 14 to permit access to and egress out of the interior of patient treatment compartment 11. Access door 14 can further include radiation shielding.

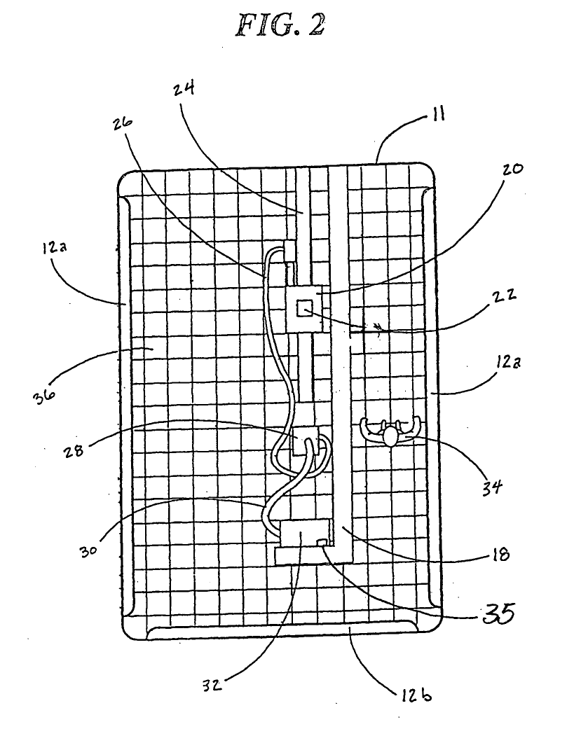

Referring now to FIG. 2, the interior of patient treatment compartment 11 is illustrated including shielded partition member 18 and treatment device 20. Treatment device 20 is preferably a radiation treatment device capable of emitting radiation used in connection with radiation therapy. In one embodiment, treatment device 20 of the present invention provides dosage of up to about 1000 centigray (CGy) (1 centigray=1 Rad) or up to about 1000 Roentgen (R). In order to protect the practitioner 34 treating the patient from repeated exposure to the radiation emitted from treatment device 20, the shielded partition member 18 is secured within patient treatment compartment 11 proximate to treatment device 20.

Shielded partition member 18 is preferably constructed from materials well known in the art to reduce or prevent exposure to radiation. Such materials include, but are not limited to, lead, aluminum, alloys of lead, polymers (such as acrylic), concrete, and fiberglass. Shielded partition member 18 can also be mounted so as to be movably positionable within patient treatment compartment 11. Shielded partition member 18 preferably extends from the floor 36 to a ceiling (not shown) of the patient treatment compartment 11 so as to prevent as much radiation as possible from interacting with body of practitioner 34. However, shielded partition member 18 need not extend along the entire distance between the floor 36 and the ceiling, but rather from the floor 36 to a height sufficient to protect the user 34.

Treatment device 20 is movably mounted on or connected to a mounting assembly comprising a stand 22, such as a tube stand, wherein the treatment device 20 may be disposed in an array of vertical positions relative to the patient. The mounting assembly further comprises a horizontal mount 24 which may be connected to the treatment device 20 and be structured to provide for the positioning of the treatment device 20 in a variety of horizontal positions relative to the patient. Stand 22 has a bottom end that is preferably flat on floor 36. Floor 36 is preferably a rubberized or formed of other material which reduces slippage so as to provide adequate traction. Stand 22 can be frictionally fit between floor 36 and the ceiling (not shown) of patient treatment compartment 11 or secured in any manner well known in the art to substantially prevent or severely restrict movement of treatment device 20, once disposed in the intended position. Such immobilization of the treatment device 20 is important to preserve the integrity of the calibration and provide precise delivery of radiation from treatment device 20 when the vehicle is stopped and treatment is provided to a patient.

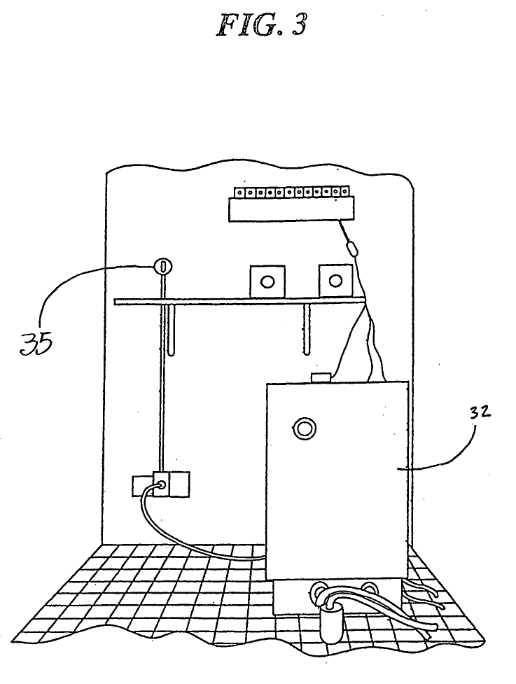

Treatment device 20 may be operationally connected to a controller 28 via cables 26. Controller 28 can be a controller appropriate to control some or all of the functions of the treatment device 20. Controller 28 is operatively connected to a control console 32 via cables 30. Control console 32 operates to regulate the functions associated with treatment device 20, including, but not limited to, power flow and duration of treatment. Isolation switch 35, as shown in FIGS. 2 and 3, is used to prevent the generators (not shown) from supplying power directly to the system. Control console 32 can also be positioned on the same side of the shielded partition 18 as user 34, for ease of operation.

In the current medical practice, it is desirable to minimize the fluctuation of both electric current, measured in amperes (amps) and electromotive force, measured in volts (V). Furthermore, current medical practice provides for a tolerance in the fluctuation of voltage and amperage during therapy. Current medical practice dictates a desired fluctuation of (+/−) 5%. This 5% fluctuation represents a maximum medically, optimally acceptable fluctuation.

In order to address this fluctuation, one embodiment provides for the radiation therapy machine of the present invention to be powered by a battery. In a preferred embodiment, the battery is a gel static battery.

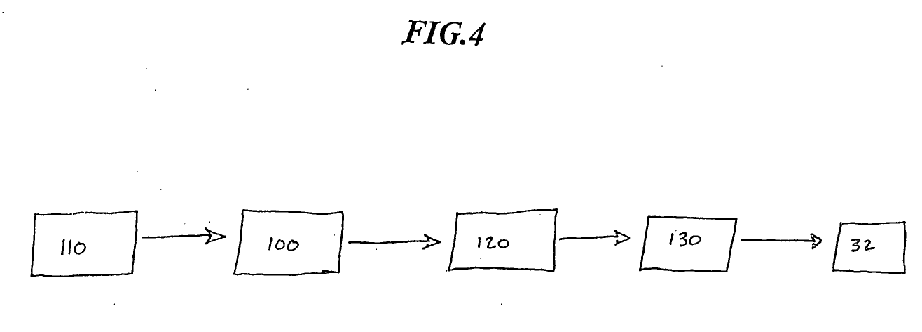

Typical generators produce an unsteady flow of electrical current, thus, as illustrated in FIG. 4, control console 32 is operatively engaged to a line conditioner and a battery isolation system 100. The arrangement of a battery 120 with an inverter 130 is to maintain an optimally stable uninterruptible supply of power to treatment device 20. Battery isolation system 100 of the present invention inverts power supplied from generators 110 to charge batteries 120 of the system. Batteries 120 then pass electrical current through an inverter 130, which then converts the electrical current to alternating current (AC) for use by treatment device 20.

Yet another embodiment may include electrical regulatory devices. These devices will be placed along the electrical circuit in a manner such that they can regulate voltage and amperage of the electric current before the current enters the radiation therapy machine.

Optionally, the regulatory device may be one or more devices well known in the art. One such device would be a voltmeter equipped with a regulator. The regulator, as is commonly known, will control the volts exiting the voltmeter to the radiation therapy machine. Additionally, such an embodiment may include an amp meter, similarly equipped with a regulator. The regulator will control the amperage exiting the regulator and supplying current to the radiation therapy machine.

The regulation of amperage and voltage becomes quite important because typical therapy may last up to 10 minutes per session. Preferably, each session is from 10 seconds to 5 minutes. In some cases, therapy will last from 30 seconds to 2 minutes. Furthermore, treatment typically is administered in repeated doses.

As better generators become available, such generators are contemplated to supply power directly to the system of the present invention without undue power fluctuation.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A mobile radiation treatment vehicle comprising:
a patient treatment compartment disposed on an interior of the vehicle,
a plurality of radiation shield members mounted on the vehicle and collectively disposed in at least partially surrounding relation to said patient treatment compartment,
a treatment device disposed within said patient treatment area and structured to emit sufficient radiation for use in radiation therapy,
said plurality of radiation shield members structured and disposed to restrict radiation emitted from said treatment device from passing from said patient treatment area to an exterior of the vehicle,
a mount assembly structured to movably support and adjustably position said treatment device within said patient treatment compartment in both a vertical direction and a horizontal direction,
a shielded partition disposed in said patient treatment compartment in any one of a plurality of radiation shielding positions between said treatment device and a user of the treatment device,
said shielded partition member selectively movable within said patient treatment area relative to said treatment device or the user thereof into any one of said plurality of shielding positions, and
said shielded partition member structured to restrict exposure of the user from radiation emitted from said treatment device during patient treatment.

2. A mobile radiation treatment vehicle as recited in claim 1 wherein said plurality of radiation shield members are formed from a shielding material that is selected from the group consisting of lead, aluminum, alloys of lead, polymers, concrete and fiber glass.

3. A mobile radiation treatment vehicle as recited in claim 1 wherein said shielded partition member is formed from shielding material that is selected from the group consisting of lead, aluminum, alloys of lead, concrete and fiber glass.

4. A mobile radiation treatment vehicle as recited in claim 1 wherein said shielded partition member extends upwardly from a floor of said patient treatment area to height sufficient to shield the user of said treatment device.

5. A mobile radiation treatment vehicle as recited in claim 4 wherein said shielded partition member extends from the floor of the vehicle upwardly to substantially adjacent a ceiling portion of the patient treatment area.

6. A mobile radiation treatment vehicle as recited in claim 1 wherein said mount assembly comprises a stand connected to said treatment device and disposed and structured to support said treatment device in any one of a plurality of vertical positions within said patient treatment area.

7. A mobile radiation treatment vehicle as recited in claim 6 wherein said mount assembly comprises a horizontal mount connected to said treatment device and disposed and structured to support said treatment device in any one of a plurality of horizontal positions.

8. A mobile radiation treatment vehicle as recited in claim 7 wherein said horizontal mount and said stand are cooperatively structured to be concurrently connected to said treatment device.

9. A mobile radiation treatment vehicle as recited in claim 7 wherein said horizontal mount has an elongated configuration and is disposed to extend along at least a portion of a length of said patient treatment area.

10. A mobile radiation treatment vehicle as recited in claim 1 wherein said mount assembly comprises a horizontal mount connected to said treatment device and disposed and structured to support said treatment device in any one of a plurality of horizontal positions.

11. A mobile radiation treatment vehicle as recited in claim 10 wherein said horizontal mount has an elongated configuration and is disposed to extend along at least a portion of a length of said patient treatment area.

12. A mobile radiation treatment vehicle as recited in claim 1 further comprising a power adapter accessible from an exterior of the vehicle and structured to provide direct line electrical power to the patient treatment area independent of electrical power resources of the vehicle.

13. A mobile radiation treatment vehicle as recited in claim 1 further comprising an access door mounted on the vehicle, said access door formed from a radiation shielding material.

13. A mobile radiation treatment vehicle as recited in claim 1 further comprising a control console disposed within said patient treatment area and structured to regulate operation of the treatment device.

14. A mobile radiation treatment vehicle as recited in claim 13 wherein said control console is structured to regulate power flow to said treatment device.

15. A mobile radiation treatment vehicle as recited in claim 14 wherein said control console is further structured to determine time duration of treatment of a patient by said treatment device.

16. A method for providing radiation therapy comprising:
preparing a mobile radiation treatment vehicle,
including a patient treatment compartment disposed on the interior of the vehicle,
including a plurality of radiation shield members collectively disposed in at least partially surrounding relation to the patient treatment compartment,
providing a treatment device within the patient treatment compartment,
structuring the treatment device to emit sufficient radiation for use in radiation therapy on a patient,
structuring the plurality of radiation shield members to restrict radiation emitted from the treatment device from passing from the patient treatment area to an exterior of the vehicle,
supporting and adjustably positioning the treatment device within the patient treatment area in both a vertical direction and a horizontal direction,
including a shielded partition member within the patient treatment compartment,
disposing the shielded partition member in any one of a plurality of radiation shielding positions between the treatment device and a user of the treatment device,
selectively moving the shielded partition member within the partition treatment area relative to the treatment device or the user into any one of the plurality of shielding positions, and
restricting exposure of the user from the radiation emitted from the treatment device during patient treatment.

17. A method as recited in claim 16 comprising extending the shielded partition member from a floor of the patient treatment area upwardly to a height sufficient to shield the user of the treatment device.

18. A method as recited in claim 16 further comprising supporting the treatment device in any one of a plurality of vertical positions.

19. A method as recited in claim 18 further comprising supporting the treatment device in any one of a plurality of horizontal positions.
